(19) United States
(12) Patent Application Publication
(10) Pub. No.: US 2005/0119621 A1
(43) Pub. Date: Jun. 2, 2005

(54) Portable medicament jet-spraying and injection apparatus

(76) Inventor: Gi-Hwan Bang, Seong-Nam City (KR)

Correspondence Address: FLESHNER & KIM, LLP, P.O. BOX 221200, CHANTILLY, VA, 20153, US

(21) Appl. No.: 10/827,443

(22) Filed: Apr. 20, 2004

(30) Foreign Application Priority Data
Nov. 28, 2003 (KR) 10-2003-85432

(51) Int. Cl. A61M 5/30

(52) U.S. Cl. 604/187

(57) ABSTRACT

The invention is related to providing a potable medicament jet spraying and injection apparatus for enabling gel typed semi-solid or liquidized medicament to be easily charged thereinto and for douching and/or injecting into a special region of a human body in a jet-spraying manner, using it. The portable medicament jet spraying and injection apparatus comprises a cylinder including a jet-spraying nozzle having a predetermined depth portion at the forehead end thereof, at its lowest end of which a cutter is mounted, a loading portion formed to receive the gel typed semi-solid or liquidized medicament and a hollow portion formed adjacent the loading portion surround the circumference of the jet-spraying nozzle; a piston portion fixed on a predetermined position to seal the gel typed semi-solid or liquidized medicament to be charged into the loading portion, which makes the loading portion to be formed as a separate chamber; and a push rod fitted into the cylinder spaced away in a certain distance from the piston portion and for pushing the piston portion toward the jet-spraying nozzle upon being pressed by external force.

### BACKGROUND OF THE INVENTION

The invention is related to providing a medicine injection device, and particularly, to providing a portable medicament jet-spraying and injection apparatus including a loading portion subject to receiving a predetermined gel typed semisolid or liquidized medicament and for douching the special regions of a human body such as vagina, anal region, etc. and injecting it thereinto in a jet-spraying manner, if necessary.

Prior Arts

A modern is being changed into a society of complex and busy everyday life and experiences to be continuingly kept. In order to be easily adapted to the modern life, there has been developed various tools or devices to consult the convenience of everyday life. Nevertheless, it is noted that it lacks of the development of a conveniently sized tool or device related to the hygiene and/or for the contraception of women or the insertion into a special region of a human body.

In other words, it is important to be careful of health elements such as the hygiene of the genital organ and vagina of women and the insertion of the contraceptive pill thereinto, the cleanliness maintaining of anal region, etc. in order to prevent diseases. But, a series of hygiene works in inserting the contraceptive pill into the human body or administrating an enema to a person have been done using hands, which is not easy and troublesome.

As it is already well known, the genital organ, vagina and anal region of human body are very sensitive and weak portions, so that their treatment or processing requires a larger carefulness. Particularly, it must be given a lot of attention to the insertion of lubricant or contraceptive pill for the sexual intercourse and medicament liquid for the treatment and prevention of the sexual disease into the genital organ or vagina and to the rectal injection of the enema.

Until now, there has been not known any device or tool that makes it convenient to douche a special region of human body such as genital organ, anal region, etc. and/or to inject a predetermined medicament thereinto. That is because the device has various limitations to ifs use including the portability that is easy to carry.

A typical technology is disclosed in Japanese Laid-Open Patent Publication No. 2001-187151, which is related to a lubricant injection device that is easy to use. The lubricant injection device comprises a plastic cylinder including an outlet port of lubricant formed at its front end and a push rod fitted into the cylinder with lubricant being charged in the front portion. The lubricant injection device enables lubricant to be applied to sexual organ or vagina in a shot time. It also removes impediments caused by a mechanical contact with corresponding sensitive regions and reduces metal burden on its safe use. And also, the lubricant injection device is easy to carry and convenient.

But, the lubricant injection device has no any countermeasure to the leakage of lubricant charged in the front portion of the cylinder, which means that it needs a lot of carefulness upon its usage. Furthermore, the lubricant injection device is disclosed as a syringe that can be inserted into a special region of human body, but it has a disadvantage in that due to its structural simplicity its use may be limited.

In light of these points, it is preferable if a medicine injection apparatus is easy to carry, safe to use and convenient to use a predetermined medical liquid or medicine in a quick time anywhere in order to douche a special region of human body and/or inject thereinto.

An object of the invention is to provide a portable medicament jet spraying and injection apparatus including a loader or container with a predetermined medicament being charged therein to douche a special region of human body and/or inject the medicament thereinto in a jet-spraying manner.

Another object of the invention is to provide a portable medicament jet spraying and injection apparatus including a loading portion or container of a gel typed capsule structure with a semisolid or liquidized medicament being charged therein to douche a special region of human body and/or inject the medicament thereinto in a jet-spraying manner.

Another object of the invention is to provide a portable medicament jet spraying and injection apparatus including a cap portion formed to secure a loading portion and prevent the leakage of liquidized medicament charged therein and a push rod separated therefrom to douche a special region of human body and/or inject the medicament thereinto in a jet-spraying manner.

### SUMMARY OF THE INVENTION

According to one embodiment of the invention, a portable medicament jet spraying and injection apparatus comprises a cylinder including a jet-spraying nozzle having a predetermined depth portion at the front end thereof, at the lowest end of which a cutting portion or cutter is mounted, a loading means or container formed to receive gel typed semi-solid or liquidized medicament and a hollow portion formed adjacent the loading means surround the circumference of the jet-spraying nozzle; a piston portion fixed on a predetermined position to seal the gel typed semisolid or liquidized medicament to be charged into the loading means, which makes the loading means to be formed as a separate chamber; and a push rod fitted into the cylinder spaced away in a certain distance from the piston portion and for pushing the piston portion toward the jet-spraying nozzle upon being pressed by external force.

According to another embodiment of the invention, a portable medicament jet spraying and injection apparatus comprises a cap portion of various forms including a jet-spraying nozzle formed to have a predetermined depth portion at its front end, a loading means enabling gel typed semi-solid or liquidized medicament to be charged, a coupling portion extended downward from the outer circumference of the loading means, in the inner portion of which a threaded portion is formed and a membrane member attached to seal the lower opening of the loading means and to be easy to be broken; a cylinder body separated from the cap portion and including a threaded portion formed at its upper portion to be engaged with the coupling portion and a push rod including supporting means for supporting it to be received therein; a piston portion pushed into the loading means from the cylinder body to enable the membrane member to be broken so that gel typed semi-solid or liquidized medicament is discharged from the loading means; and a push rod fitted into the cylinder spaced away in a certain distance from the piston portion and for pushing the piston portion into the loading means to break the membrane member upon being pressed by external force.

### BRIEF DESCRIPTION OF THE DRAWINGS

FIG. 1 is an exploded perspective view illustrating a configuration of a portable medicament jet spraying and injection apparatus according to one embodiment of the invention;

FIG. 2 is a cross-sectional view illustrating the important portion of a portable medicament jet spraying and injection apparatus according to one embodiment of the invention;

FIG. 3 is an enlarged cross-sectional view illustrating the important portion of a portable medicament jet spraying and injection apparatus according to one embodiment of the invention;

FIG. 4 is a cross-sectional view illustrating the operating of a portable medicament jet spraying and injection apparatus according to one embodiment of the invention;

FIG. 5 is a cross-sectional view illustrating a coupling relation between a piston portion and a push rod of a portable medicament jet spraying and injection apparatus according to one embodiment of the invention;

FIG. 6 is an enlarged cross-sectional view illustrating the important portion of a portable medicament jet spraying and injection apparatus according to another embodiment of the invention;

FIG. 7 is a cross-sectional view illustrating the operating of a portable medicament jet spraying and injection apparatus according to another embodiment of the invention;

FIG. 8 is an enlarged cross-sectional view illustrating a cylindrical cap portion of a portable medicament jet spraying and injection apparatus according to another embodiment of the invention;

FIG. 9 is an enlarged cross-sectional view illustrating a semi-circular cap portion of a portable medicament jet spraying and injection apparatus according to another embodiment of the invention;

FIG. 10 is an enlarged cross-sectional view illustrating a triangle cap portion of a portable medicament jet spraying and injection apparatus according to another embodiment of the invention; and,

FIG. 11 is an enlarged cross-sectional view illustrating a trapezoidal cap portion of a portable medicament jet spraying and injection apparatus according to another embodiment of the invention.

### DETAILED DESCRIPTION OF THE INVENTION

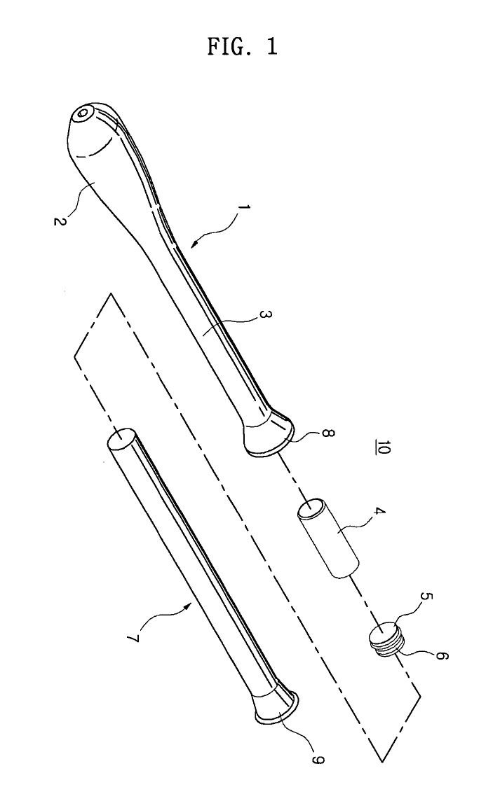

According to one embodiment of the invention, as shown in FIG. 1, a portable medicament jet spraying and injection apparatus 10 comprises a cylinder 1 including an insertion 2 expanded in an oval or circular shape at its front portion, a dept portion extended in a straight line from the lower of the insertion 2 and a first expanding portion 8 expanded outside toward its lower end at a funnel shape. The cylinder 1 has a hollow formed in the same diameter through its all length portion, in which a loading portion is formed adjacent a front end to receive a gel typed gelatin capsule 4.

A piston portion 5 is positioned adjacent the loading portion as described below in detail. The piston portion 5 includes a plurality of grooves 6 formed surround the outer circumference thereof. Some or any one of the grooves 6 is coupled with a fixing portion formed in the cylinder 1 to determine the fixing position of the piston portion. The other grooves 6 comprise a seal member such as a rubber-inserted thereinto to seal a lower opening of the loading portion. A push rod 7 is separated from the piston portion 5 and includes a second expanding portion 9 formed at a lower end to couple with the first expanding portion 8.

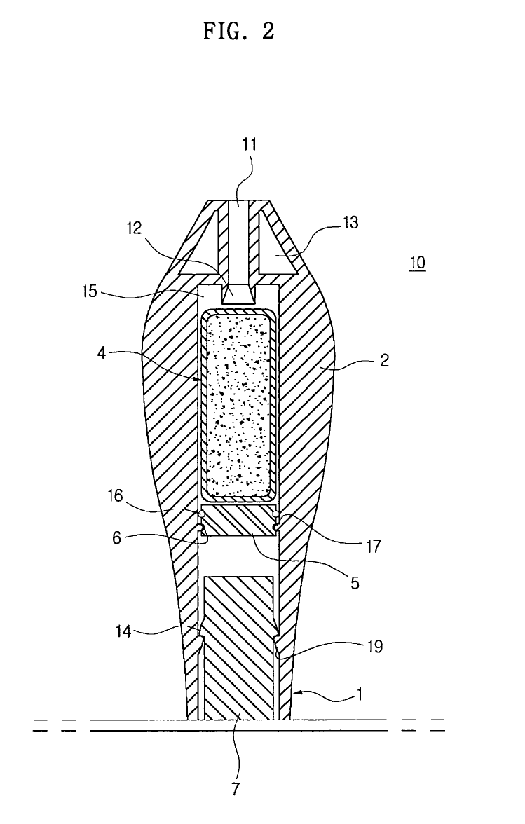

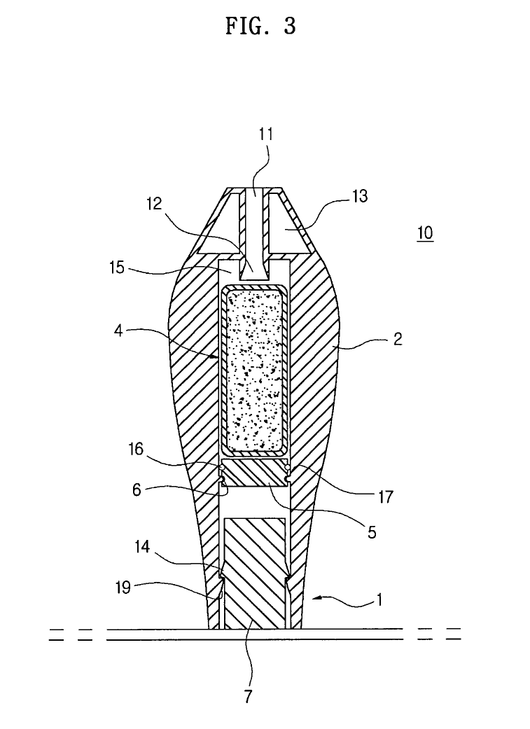

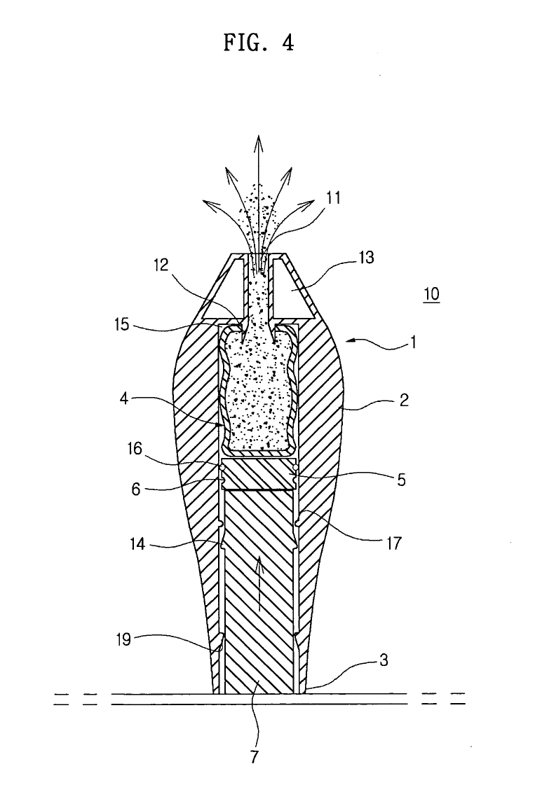

Referring to FIGS. 2, 3 and 4 for the purpose of explaining the invention more in detail, FIG. 2 is a whole cross-sectional view; FIG. 3 is a partly enlarged cross-sectional view and FIG. 4 is a cross-sectional view illustrating the operating of a portable medicament jet spraying and injection apparatus.

A cylinder 1 includes an insertion 2 expanded in a circular or a streamline form of an oval somewhat over a length portion 3, so that it is easy to be inserted into a special region of human body. A jet-spraying nozzle 11 is formed to have a certain depth portion that is extended downward from the front. A cutter 12 is constituted as a blade integrated with the jet-spraying nozzle 11. A hollow portion 13 is formed to surround the circumference of the jet-spraying nozzle 11 adjacent thereto, so that it facilitates the insertion of medicine liquid into a loading portion 15 as described in detail below.

The loading portion 11 is formed as a hollow space for receiving a gelatin capsule 4 of a semisolid type or medicament of a medicine liquid type. For example, the gel typed capsule 4 having a predetermined medicament is previously charged into the loading portion 15 in the cylinder 1 through the inner hollow space of the length portion 3 from the first expanding portion 8. The loading portion 15 has a volume to be determined by the piston portion 5.

The piston portion 5 is a disk plate of a predetermined thickness comprising a plurality of grooves 6. For example, the grooves 6 includes upper groove into which a sealing member 16 such as elastic rubber is inserted and lower groove into which an annular projector 17 formed in part or all over around the inner circumference of the cylinder 1 is fitted.

The push rod 7 is fitted into the hollow space of the length portion 3 to be positioned at a predetermined position therein with being separated from the piston portion 5. In other words, from around the inner circumference of the cylinder 1, a stopper 9 is projected, the upper of which is formed as a rectangular step portion to its inner wall surface and the lower of which is formed as a slanting portion sloped from the step portion. The push rod 7 includes a rib 14 extended all over from the outer circumference thereof to engage with the step portion of the stopper 19. As the push rod 7 is inserted into the length portion 3, the rib 14 rides on and then passes over the stopper 19 to be supported at a predetermined position.

As shown in FIGS. 3 and 4, the portable medicament jet-spraying and injection apparatus 10 enables the gelatin capsule 4 to be positioned in the loading portion 15 in which the medicament is charged. As the push rod 7 is pushed inward contacting with the piston portion 5. The cutter 12 breaks out the upper portion of the gelatin capsule 4, so that the medicament therein is injected into or distributed over the special region of human body through the jet-spraying nozzle 11. Herein, it is noted that the medicament may be douching material or lubricant.

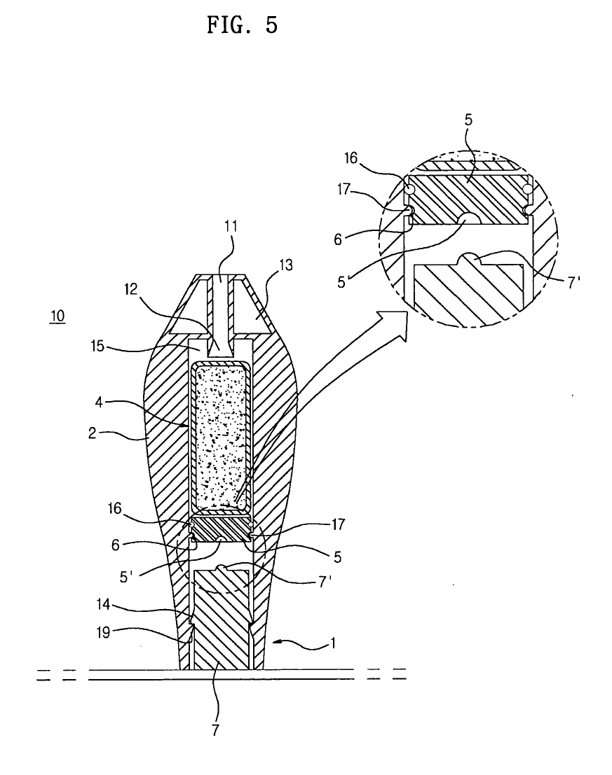

On the other hand, another configuration of a piston portion 5 and a push rod 7 is shown in FIG. 5. The piston portion 5 includes a groove 5′ formed at the lower center thereof, and the push rod 7 includes a projection 7′ projected upward from the upper surface thereof to be fitted into the groove 5′. The projection 7′ serves as a handler to position the piston portion 5 at a certain position with being coupled with the groove 5′, so that the medicament of liquid or semisolid type is charged into the loading portion 15. After the medicament is completely charged, the push rod 7 is forcedly distorted from the piston portion 5 to be separated from each other. Of course, the groove 5′ and the projection 7′ each includes a threaded portion formed on the inner and outer portions thereof to be coupled to each other.

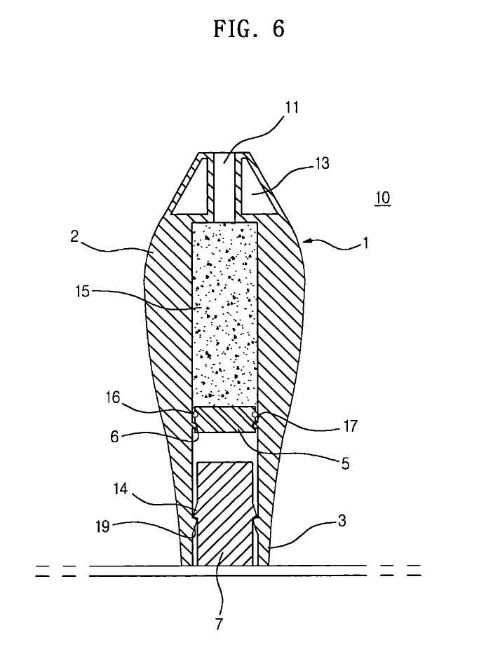

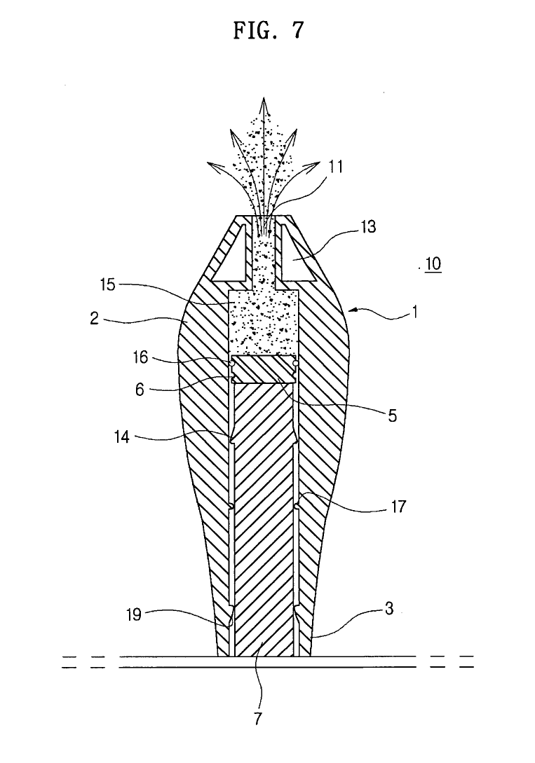

Another embodiment of the invention is shown in FIGS. 6 and 7. According to this embodiment, a portable medicament jet-spraying and injection apparatus 10 is the same as the first embodiment except a configuration that a cutter 12 is not provided on a jet-spraying nozzle 11. Therefore, further detailed explanation may be deleted to avoid the overlap. Only, it is noted that a piston portion 5 having a position previously fixed to have a predetermined volume defines a loading portion 15. The charging of medicament into the loading portion 15 is to form a certain space using the piston portion 5 and the push rod 7, properly, in order to insert an amount of medicament with the jet-spraying nozzle 11 being not sealed at the upper end. Otherwise, an amount of medicament may be charged into the loading portion 15 using a needle passed through a hollow portion 13.

As described above, the loading portion 15 is constituted as an independent chamber by the piston portion 5 with being completely sealed. As the pushing rod 7 is pushed against the piston portion 5, the medicament charged into the loading portion 15 is injected into or distributed over a special region of human body through the jet-spraying nozzle 11 for establishing a predetermined purpose. The circular or oval insertion 2 is safe for human body. Also, the cylinder 1 having the medicament is safe and easy to carry.

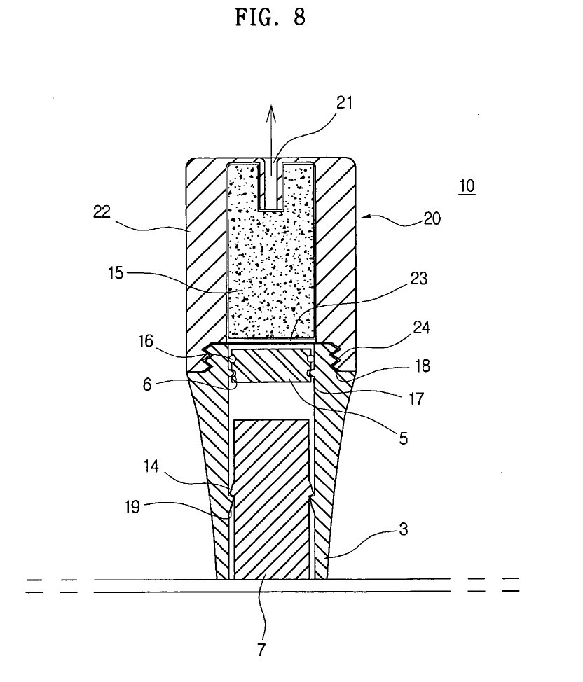

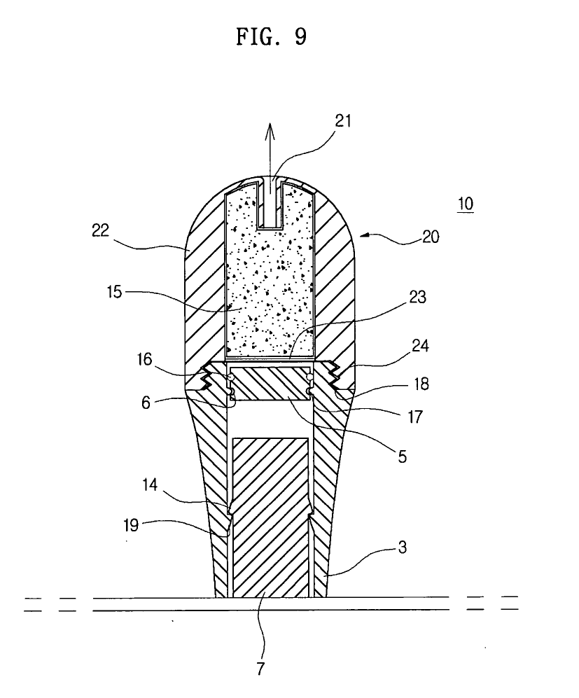

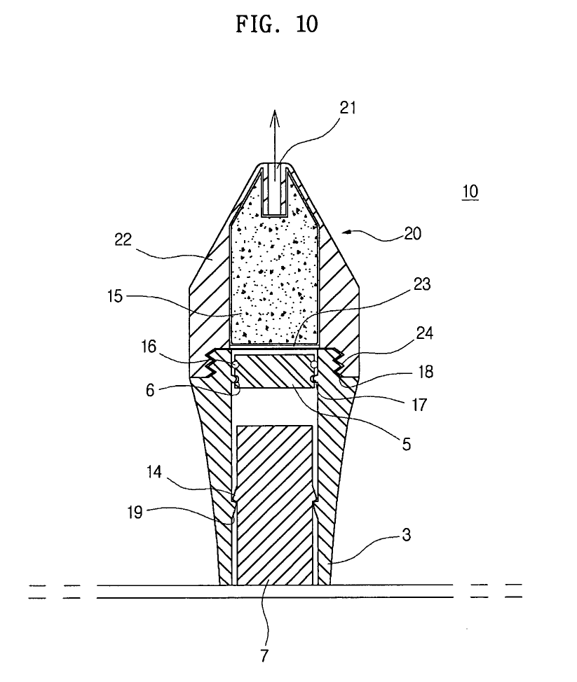

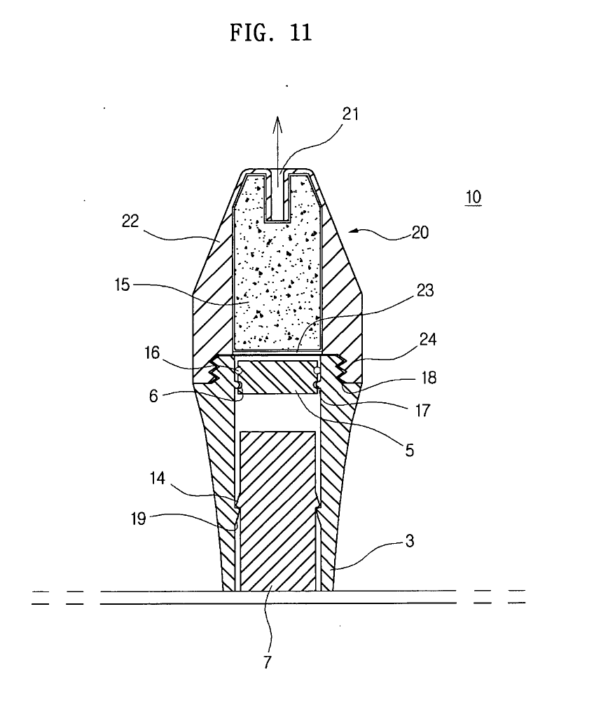

Another embodiment of the invention is shown in FIGS. 8, 9, 10 and 11, in which the FIG. 8 is a view of a quadrilateral cap portion, FIG. 9 is a view of a semicircular cap portion, FIG. 10 is a view of a triangular cap portion and FIG. 10 is a view of a trapezoidal cap portion.

According to this embodiment, a potable medicament jet-spraying and injection apparatus 10 comprises a cylinder 1 and a cap portion 20, in which the cylinder 1 includes a length portion 3 and its inner structure such as a stopper and a piston portion 5 same as the first embodiment. Therefore, the detailed description to the same elements or parts referenced by same numbers may be deleted.

Part unlike the first embodiment is that the cylinder 1 is coupled with a cap portion 20 instead of an insertion 2. The cap portion 20 includes a male engagement 18 forming a plurality of threads around the upper circumference thereof.

The cap portion 20 includes a jet-spraying nozzle 21 extended downward in a predetermined depth from the upper end thereof and a loading portion 22 having a hollow space for receiving medicament of a semisolid or liquid type. Also, the cap portion 20 includes a membrane member 23 for sealing the lower opening thereof and a female engagement 24 extended downward from the lower portion thereof, on the inner circumference of which a threaded portion is formed to couple with the male engagement 18. Therefore, the cap portion 20 has medicament in the loading portion 15 with the upper end of the jet-spraying nozzle 11 being sealed by an adhesive tape, etc and the lower opening being closed by the membrane member 23.

Next, the cylinder 1 is assembled so that the piston portion 5 is positioned adjacent the upper portion thereof by coupling an annular projector 17 formed on the inner portion of the cylinder 1 with the groove 6 thereof, and the push rod 7 is positioned in the hollow space thereof in a manner to engage a stopper 19 formed on the inner portion of the cylinder 1 with a rib 14 of the push rod 7.

Such like cap portion 20 can be constructed as various shapes to charge medicaments including douching agents or lubricants thereinto for the purpose of many uses. Therefore, as the pushing rod 7 is pushed against the piston portion 5 to break the membrane member 23, these cap portions 20 fixed on the upper male engagement 24 injects the medicament charged in the loading portion 22 into or distributes over a special region of human body through the jet-spraying nozzle 21 like the above embodiments.

As described above, the invention enables the effective charging of medicament of a semisolid or liquid type into an independent hollow space thereof and the rapid injection and distribution of medicaments thanks to their use convenience. Furthermore, the invention enables a special region of human body such as genital organ, vagina, anal region, etc. to be douched for the cleaning purpose or to be injected into a special region of human body in a jet-spraying manner for the purpose of the contraception or the protection of venereal disease, effectively.

1. A portable medicament jet spraying and injection apparatus for douching/injecting medicament of a semi-solid or liquid type into a special region of human body comprising:
a cylinder including a jet-spraying nozzle having a predetermined depth portion at the front end thereof, a loading means or container formed to receive gel typed semi-solid or liquidized medicament and a hollow portion formed adjacent the loading means surround the circumference of the jet-spraying nozzle;
a piston portion fixed on a predetermined position to seal the gel typed semi-solid or liquidized medicament to be charged into the loading means, which makes the loading means to be formed as a separate chamber; and
a push rod fitted into the cylinder spaced away in a certain distance from the piston portion and for pushing the piston portion toward the jet-spraying nozzle upon being pressed by external force.

2. The portable medicament jet spraying and injection apparatus as claimed in claim 1, in which:
the jet-spraying nozzle includes a cutter mounted at the lower edge portion of the depth portion that is extended downward from the upper portion of the cylinder.

3. The portable medicament jet spraying and injection apparatus as claimed in claim 1, in which:
the loading means includes a hollow space independently divided to receive the medicament of a gel typed capsule or a liquid type and to inject the medicament outward through the jet-spraying nozzle.

4. The portable medicament jet spraying and injection apparatus as claimed in claim 1, in which:
the piston portion includes at least one groove formed around the outer circumference thereof, in which a sealing member is fitted in one groove to be movable in the cylinder and an annular projector is coupled with another groove.

5. The portable medicament jet spraying and injection apparatus as claimed in claim 1, in which:
the push rod includes a rib projected all over from the outer circumference thereof, and the cylinder includes a stopper projected from around the inner circumference thereof, so that the rib and step portion enables the rod to be supported at a certain position of the cylinder.

6. The portable medicament jet spraying and injection apparatus as claimed in claim 1, in which:
the piston includes a groove formed at the lower center thereof, and the push rod includes a projection corresponding to the groove, so that the piston and the push rod are coupled with each other.

7. A portable medicament jet spraying and injection apparatus for douching/injecting medicament of a semi-solid or liquid type into a special region of human body comprising:
a cap portion of various forms including a jet-spraying nozzle formed to have a predetermined depth portion at its front end, a loading means enabling gel typed semi-solid or liquidized medicament to be charged, a female engagement extended downward from the outer circumference of the loading means, in the inner portion of which a threaded portion is formed and a membrane member is attached to seal the lower opening of the loading means and to be easy to be broken;
a cylinder body separated from the cap portion and including a threaded portion formed at its upper portion to be engaged with the coupling portion and a push rod including supporting means for supporting it to be received therein;
a piston portion pushed into the loading means from the cylinder body to enable the membrane member to be broken so that gel typed semi-solid or liquidized medicament is discharged from the loading means; and
a push rod fitted into the cylinder spaced away in a certain distance from the piston portion and for pushing the piston portion into the loading means to break the membrane member upon being pressed by external force.

8. The portable medicament jet spraying and injection apparatus as claimed in claim 7, in which:
the cap portion is constructed as various shapes such as a quadrilateral form, a semicircular form, a triangular form and a trapezoidal form.

9. The portable medicament jet spraying and injection apparatus as claimed in claim 7, in which:
the piston portion includes a plurality of grooves formed around the outer circumference thereof, into some of which sealing members is inserted and with the others of annular projectors are coupled.

10. The portable medicament jet spraying and injection apparatus as claimed in claim 1, in which:
the push rod includes a rib projected all over from the outer circumference thereof, and the cylinder includes a stopper projected from around the inner circumference thereof to be not separated from each other upon being assembled.
